(19) United States
(12) Patent Application Publication
(10) Pub. No.: US 2014/0236062 A1
(43) Pub. Date: Aug. 21, 2014

(54) BLOOD FLOW CONDUCIVE BRACE

(71) Applicant: William Reynolds, West Berlin, NJ (US)

(72) Inventor: William Reynolds, West Berlin, NJ (US)

(21) Appl. No.: 14/181,184

(22) Filed: Feb. 14, 2014

Related U.S. Application Data
(60) Provisional application No. 61/765,155, filed on Feb. 15, 2013.

(51) Int. Cl. A61F 5/01

(52) U.S. Cl. CPC A61F 5/013; A61F 5/0102
USPC 602/20; 602/5

(57) ABSTRACT

A limb brace used to promote blood flow is provided. The limb brace may be made of a semi-rigid skeleton made of a plurality of intersecting ribs. A padded lining may surround the skeleton for comfort. The limb brace may be tubular, may be attachable to a user's upper arm.

### CROSS-REFERENCE TO RELATED APPLICATION

This application claims the benefit of priority of U.S. provisional application No. 61/765,155, filed Feb. 15, 2013, the contents of which are herein incorporated by reference.

### BACKGROUND OF THE INVENTION

The present invention relates to a brace and, more particularly, to a blood flow conducive brace.

Obstruction of nerves and the flow of blood can occur during the night due to improper sleeping positions. This can cut off the circulation or pinch nerves and cause Paresthesia, which can be very uncomfortable and may disrupt sleep.

As can be seen, there is a need for device that prevents Parethesia.

### SUMMARY OF THE INVENTION

In one aspect of the present invention, a limb brace comprises: a top end, a bottom end, an inside surface, an outside surface, a first side and a second side; a skeleton comprising a plurality of semi-rigid ribs; and a fastener releasably attaching the first side and the second side together forming a tube configured to surround a limb of a user.

In another aspect of the present invention, an apparatus comprises: a tubular limb brace sized to fit between an elbow and a shoulder of a user's arm, wherein the tubular limb brace comprises a skeleton comprising a plurality of intersecting semi-rigid ribs.

These and other features, aspects and advantages of the present invention will become better understood with reference to the following drawings, description and claims.

### BRIEF DESCRIPTION OF THE DRAWINGS

FIG. 1 is a perspective view of the present invention shown in use;

FIG. 2 is a perspective view of the present invention;

FIG. 3 is a perspective view of the present invention in the unrolled position;

FIG. 4 is a section view of the present invention, taken along line 5-5 in FIG. 2;

FIG. 5 is a front view of the present invention illustrating the attachment of the shoulder cap; and

FIG. 6 is a side view of the present invention illustrating the movement of the shoulder cap.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a limb brace used to promote blood flow. The limb brace may be made of a semi-rigid skeleton made of a plurality of intersecting ribs. A padded lining may surround the skeleton for comfort. The limb brace may be tubular, may be attachable to a user's upper arm.

The present invention may include a device that prevents nerve obstruction and allows a consistent clear path for blood flow through the limbs. The limbs may be limbs of an animal, such as a human. Thereby, the present invention may prevent Paresthesia and the discomfort that comes with it.

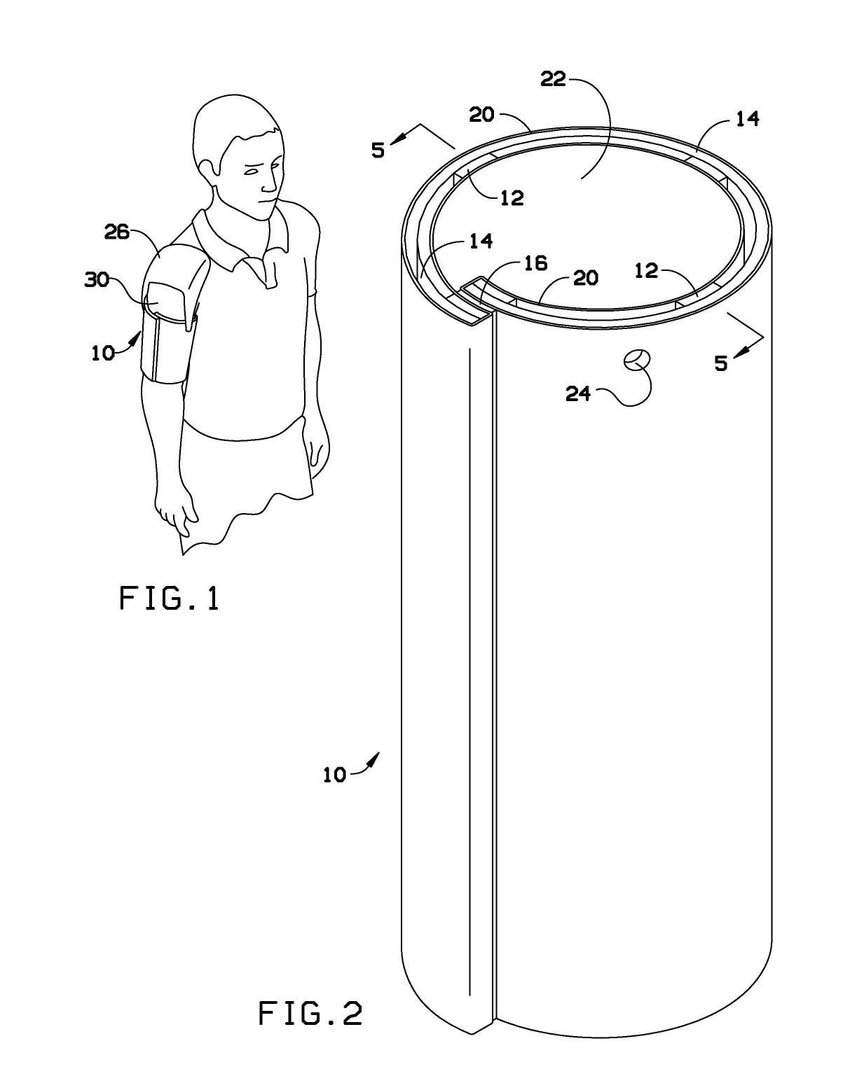

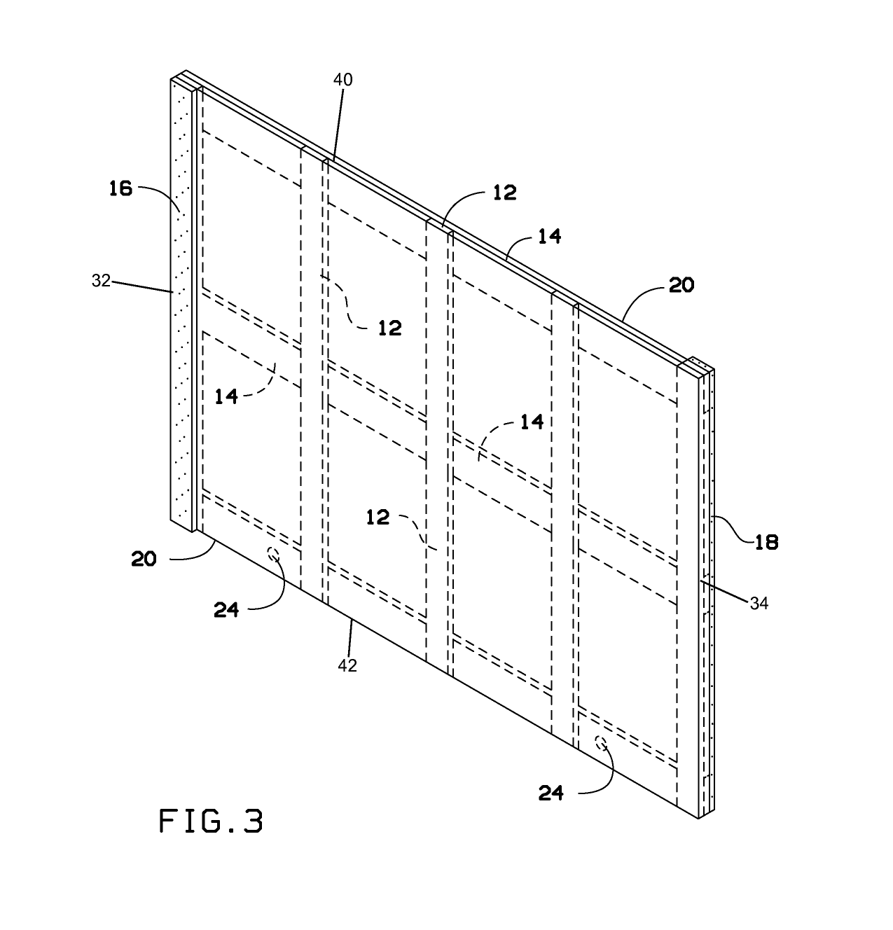

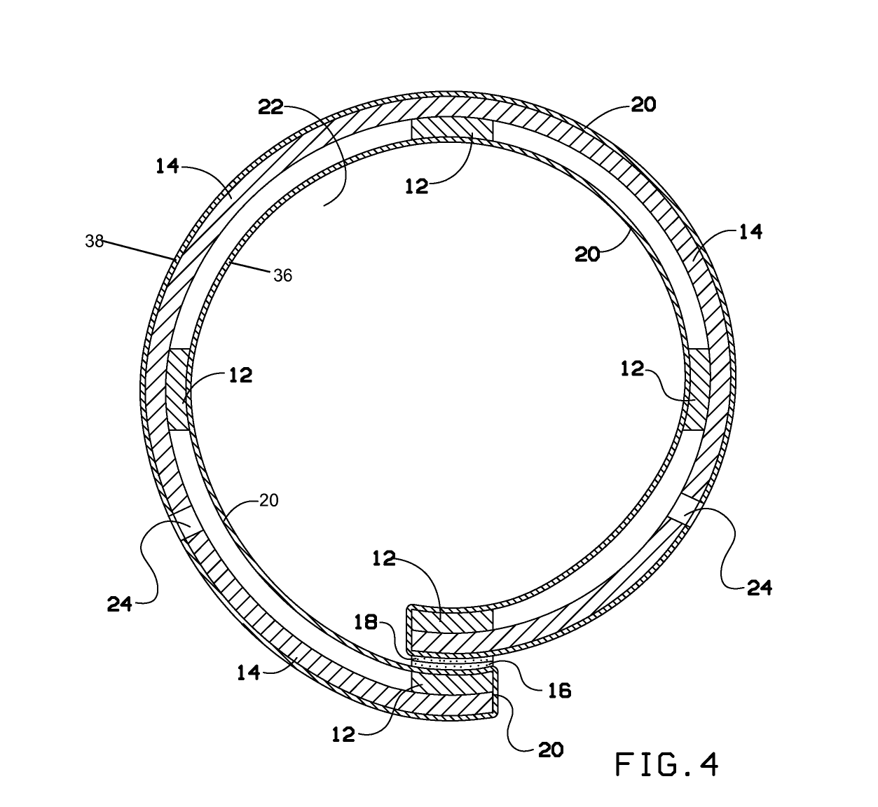

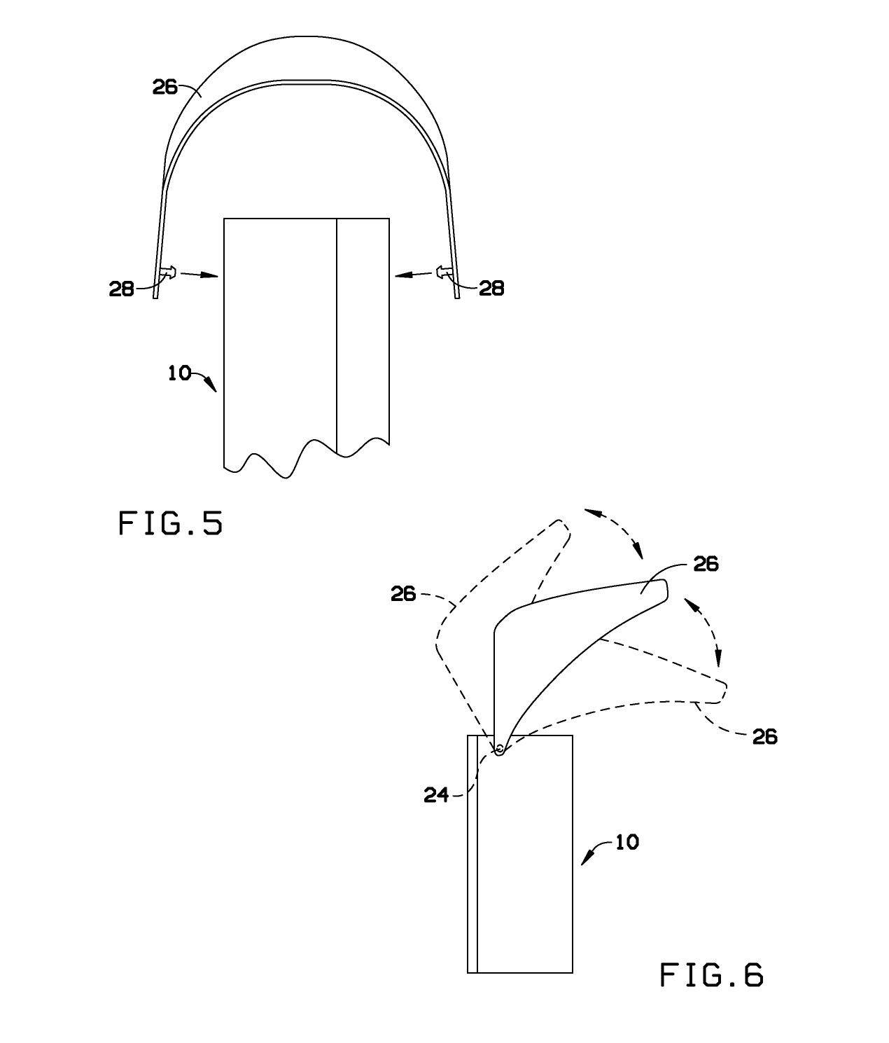

Referring now to FIGS. 1 through 6, the present invention may include a tubular arm brace 10. The brace 10 may be wrapped around and secured to a user's limbs 30, such as the upper arms of a person, adjacent to the shoulder. However, the brace 10 may also be wrapped around the upper and lower leg, as well as the lower arm. The brace 10 may be made of a semi-rigid sturdy and bendable material, such as a polymer or metal. When the brace 10 is open, the brace 10 may be rectangular in shape with a first side 32, a second side 34, an inside surface 36, an outside surface 38, a top end 40 and a bottom end 42.

In certain embodiments, the brace 10 may include a fastener. The fastener may secure the first end 32 to the second end 34 together. In certain embodiments, the fastener may include buttons, buckles, a hook and loop fastener 16, 18 and the like. For example, the first side 32 may include a loop fastener strip 16 and the second side 34 may include a hook fastener strip 18. The hook and loop fastener may be on the inside surface 36 of the first side 32 and the outside surface 38 of the second side 34. In certain embodiments, the fastener may be adjustable by including longer sheets of hook and loop fastener, adjustable straps and the like. Therefore, the size of the opening 22 formed by the fastened together brace 10 may be adjustable.

The brace 10 may further include a skeleton made of multiple ribs. As illustrated in the Figures, there may be vertical ribs 12. In certain embodiments, there may be five vertical ribs 12. The vertical ribs 12 may be made of a semi-rigid sturdy, hard and bendable material. The first end 32 and the second end 34 may both include vertical ribs 12. The skeleton may further include horizontal ribs 14. In certain embodiments, the present invention may include three horizontal ribs 14. The horizontal ribs 14 may bend around the arm and create a tube. In such embodiments, when the brace 10 is be placed and secured to the arm, the vertical ribs 12 may surround the arm of the user. The horizontal ribs 14 and the vertical ribs 12 may intersect, and thereby crisscross.

In certain embodiments, the present invention may include a padded lining 20. The padded lining 20 may form the inside surface 36 and the outside surface 38 of the brace 10. The padded lining 20 may cover the skeleton and therefore the brace 10 may be more comfortable to wear.

In certain embodiments, the brace 10 may include add-on attachments. In such embodiments, the brace 10 may include an attachment component near the top end 42 of the brace. The attachment component may include anchor holes 24, snaps, hook and loop fasteners, clips and the like. The attachment component may be used to add attachments to the brace 10.

The attachment may include a shoulder cap 26. The shoulder cap 26 may be made of a hard plastic and may be attached to the attachment component at the top end 40 of the brace 10. For example, the shoulder cap 26 may include shoulder anchors 28. The shoulder anchors 28 may be inserted into the anchor holes 24 and may thereby pivotally connect with the brace 10. The shoulder cap 26 may include a first end and a second end each containing the anchors 28, and a rounded body portion. Therefore, the rounded body portion of the shoulder cap 26 may rest on the shoulder of a user. The cap 26 may be adjusted and moved. The shoulder cap 26 may be used to redirect pressure of the weight to the skeleton of the brace 10.

The present invention may be used in the following manner. The brace may be put on a user by taking the end that the attachment device, wrapping the brace around the limb to fit, and securing the attachment devices together. The present invention may apply a small amount of pressure to the limb. If the shoulder caps are used, the user may line up the holes on the shoulder cap to the holes on the side of the brace. The user may snap plastic clips within the holes. Once the brace is on it, the shoulder cap may take weight or pressure that normally sets on the arm or shoulder. The present invention may then provide a clear path for the blood to run through a user's limbs.

The brace can be made several different ways. A skeleton frame may be made out of hard but flexible material such as plastic, metal, etc. The ribs that make up the skeleton may run vertically and horizontally. A lightly padded material may cover the outside and inner side of the brace and may be sewn together. The shoulder cap may include a curve that fits around the shoulder. The shoulder cap may be attached to the rib closest to the shoulder with pegs that go through hole in the cap and rib.

It should be understood, of course, that the foregoing relates to exemplary embodiments of the invention and that modifications may be made without departing from the spirit and scope of the invention as set forth in the following claims.

What is claimed is:

1. A limb brace comprising:
a top end, a bottom end, an inside surface, an outside surface, a first side and a second side;
a skeleton comprising a plurality of semi-rigid ribs; and
a fastener releasably attaching the first side and the second side together forming a tube configured to surround a limb of a user.

2. The limb brace of claim 1, wherein the fastener is a hook and loop fastener.

3. The limb brace of claim 2, wherein a hook fastener strip is attached to the inside surface of the first side, and a loop fastener strip is attached to the outside surface of the second side.

4. The limb brace of claim 1, wherein the diameter of the tube is adjustable.

5. The limb brace of claim 1, further comprising a padded lining covering the skeleton.

6. The limb brace of claim 1, wherein the plurality of semi-rigid ribs crisscross one another.

7. The limb brace of claim 1, wherein the plurality of semi-rigid ribs comprises a plurality of vertical ribs and a plurality of horizontal ribs, wherein the plurality of vertical ribs intersect with the plurality of horizontal ribs.

8. The limb brace of claim 1, further comprising a shoulder cap pivotally attached to the limb brace near the top end.

9. An apparatus comprising:
a tubular limb brace sized to fit between an elbow and a shoulder of a user's arm, wherein the tubular limb brace comprises a skeleton comprising a plurality of intersecting semi-rigid ribs.

10. The apparatus of claim 9, further comprising a padded lining covering the skeleton.

11. The apparatus of claim 9, wherein the plurality of semi-rigid ribs comprises a plurality of vertical ribs and a plurality of horizontal ribs, wherein the plurality of vertical ribs intersect with the plurality of horizontal ribs.

12. The apparatus of claim 9, further comprising a shoulder cap pivotally attached to the limb brace.
